(19) United States
(12) Patent Application Publication
(10) Pub. No.: US 2022/0023073 A1
(43) Pub. Date: Jan. 27, 2022

(54) GASTROINTESTINAL STENT

(71) Applicant: Kawasumi Laboratories, Inc., Oita (JP)

(72) Inventor: Noriaki SHIRAHAMA, Oita (JP)

(73) Assignee: Kawasumi Laboratories, Inc., Oita (JP)

(21) Appl. No.: 17/296,262

(22) PCT Filed: Dec. 10, 2019
(86) PCT No.: PCT/JP2019/048174

§ 371 Date: May 24, 2021

(51) Int. Cl. A61F 2/88; A61F 2/04

(52) U.S. Cl. CPC A61F 2/88; A61F 2002/045; A61F 2/04

(57) ABSTRACT

Provided is a gastrointestinal stent that can be placed accurately at a placement target site of the gastrointestinal tract. A gastrointestinal stent (large intestine stent 1) is a gastrointestinal stent that is placed inside the gastrointestinal tract (large intestine C) and has a tubular shape. The gastrointestinal stent comprises: a skeleton portion (11) capable of expanding and contracting in a radial direction substantially perpendicular to an axial direction; and a conversion section (restraint string 13) capable of converting one portion (rear end portion 11b) of the skeleton portion in the axial direction from the contracted state to the expanded state while keeping the other portion (front end portion 11a) of the skeleton portion in the contracted state.

### TECHNICAL FIELD

The present invention relates to a gastrointestinal stent.

### BACKGROUND ART

Conventionally, there have been known gastrointestinal stents that are placed at a stenosis site or an occluded site generated in a digestive system lumen such as esophagus, stomach, small intestine, large intestine, and bile duct (hereinafter, referred to as “gastrointestinal tract”) and expand a diameter of a lesion site to maintain an opening state of the gastrointestinal tract (e.g. see Patent Document 1).

### PRIOR ART DOCUMENT

### Patent Document

Patent Document 1: Japanese Patent No. 4651943

### SUMMARY OF THE INVENTION

### Technical Problem

Incidentally, the gastrointestinal stent is delivered to a placement target site by a stent placement system, and is accommodated in a sheath of the stent placement system while contracting in a radial direction and extending in an axial direction. Thus, it is difficult to smoothly release the stent from the sheath, and it is difficult to accurately place the gastrointestinal stent at the placement target site in the gastrointestinal tract because the gastrointestinal stent is shortened in the axial direction when the gastrointestinal stent is released from the sheath and a skeleton portion of the gastrointestinal stent expands. In particular, a gastrointestinal stent having a skeleton portion around which a wire rod is spirally wound has a high reduction rate (shortening), so that the aforementioned problem is remarkable.

An object of the present invention is to provide a gastrointestinal stent that can be accurately placed at a placement target site of a gastrointestinal tract.

### Solution to Problem

The gastrointestinal stent according to the present invention, which is placed in a gastrointestinal tract, and includes a skeleton portion having a cylindrical shape and capable of expanding and contracting in a radial direction substantially perpendicular to an axial direction, and a conversion section that can, while maintaining one part of the skeleton portion in an axial direction in a contracted state, convert an other part from the contracted state to an expanded state.

### Advantageous Effect of the Invention

According to the present invention, a gastrointestinal stent can be accurately placed at a placement target site of a gastrointestinal tract.

### BRIEF DESCRIPTION OF THE DRAWINGS

FIG. 1A is a diagram illustrating a configuration of a stent placement system according to an embodiment.

FIG. 1B is a diagram illustrating a configuration of the stent placement system according to the embodiment.

FIG. 2 is an external perspective view schematically illustrating a configuration of a large intestine stent according to the embodiment.

FIG. 3 is a diagram illustrating a state that a restraint string is attached to a second skeleton portion.

FIG. 4A is a diagram illustrating a state change during placement of the large intestine stent.

FIG. 4B is a diagram illustrating a state change during placement of the large intestine stent.

FIG. 4C is a diagram illustrating a state change during placement of the large intestine stent.

FIG. 4D is a diagram illustrating a state change during placement of the large intestine stent.

### DESCRIPTION OF THE EMBODIMENT

Hereinafter, the embodiment of the present invention will be explained in detail with reference to the figures.

In the present embodiment, as an example of the present invention, a large intestine stent 1 will be explained, which is placed in a large intestine for the purpose of treating occlusion (stenosis) by widening a lesion site of a large intestine (e.g. occluded site or stenosis site of large intestine) outward in a radial direction.

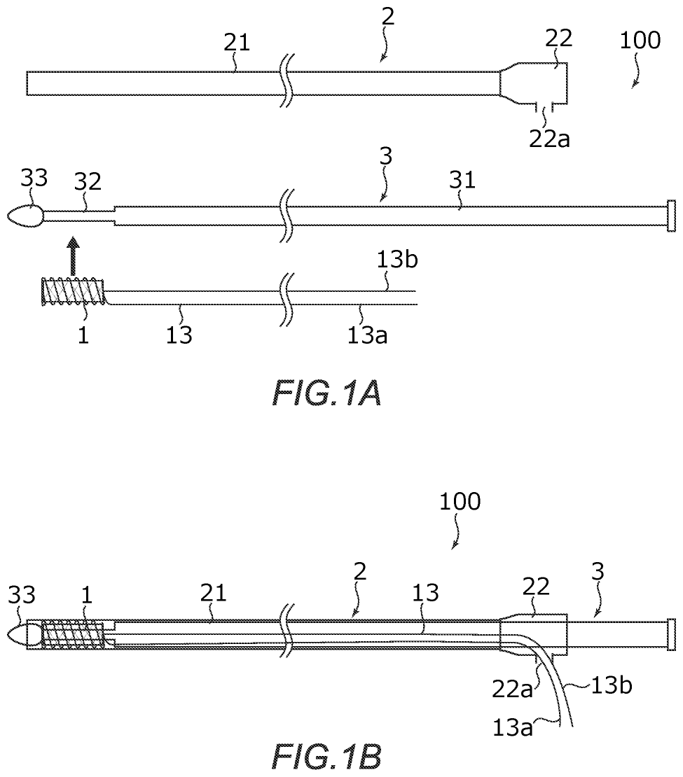

FIG. 1A and FIG. 1B are diagrams illustrating a configuration of a stent placement system 100. FIG. 1A illustrates a state that the stent placement system 100 is disassembled, and FIG. 1B illustrates a state that the stent placement system 100 is assembled. FIG. 1A and FIG. 1B schematically illustrate a size (length, diameter, etc.), a shape, and the like of each member constituting the stent placement system 100 for the purpose of facilitating understanding of the invention.

When the large intestine stent 1 is placed in the large intestine, the stent placement system 100 is used e.g. so as to be inserted into a forceps hole of an endoscope. As illustrated in FIG. 1A and FIG. 1B, the stent placement system 100 includes a tubular sheath 2, an inner rod 3 disposed inside the sheath 2 and configured to be capable of advancing and retreating along an axial direction (longitudinal direction) of the sheath 2, and the large intestine stent 1 accommodated in the sheath 2 in a contracted state so as to be expandable in the radial direction.

The sheath 2 has e.g. a tubular sheath main part 21 made of a flexible material, and a hub 22 disposed on a proximal end side (right side in FIG. 1A and FIG. 1B) of the sheath main part 21 and for fixing and releasing the inner rod 3 to or from the sheath main part 21.

The inner rod 3 has e.g. a bar-shaped rod main part 31, a holding portion 32 formed so as to have a diameter smaller than of the rod main part 31 and for holding the large intestine stent 1 in a contracted state, and a front end tip 33 disposed on a front end portion (distal end portion) of the inner rod 3.

Although not illustrated, on the rod main part 31, the holding portion 32, and the front end tip 33, a guide wire lumen through which a guide wire passes, a trigger wire lumen through which a trigger wire passes for expanding the contracted large intestine stent 1 on a lesion, and the like are formed along an axial direction of the inner rod 3.

In addition, the rod main part 31, the holding portion 32 and the front end tip 33 are made of e.g. various materials having moderate hardness and flexibility such as a resin and a metal, but their detailed explanation is omitted herein.

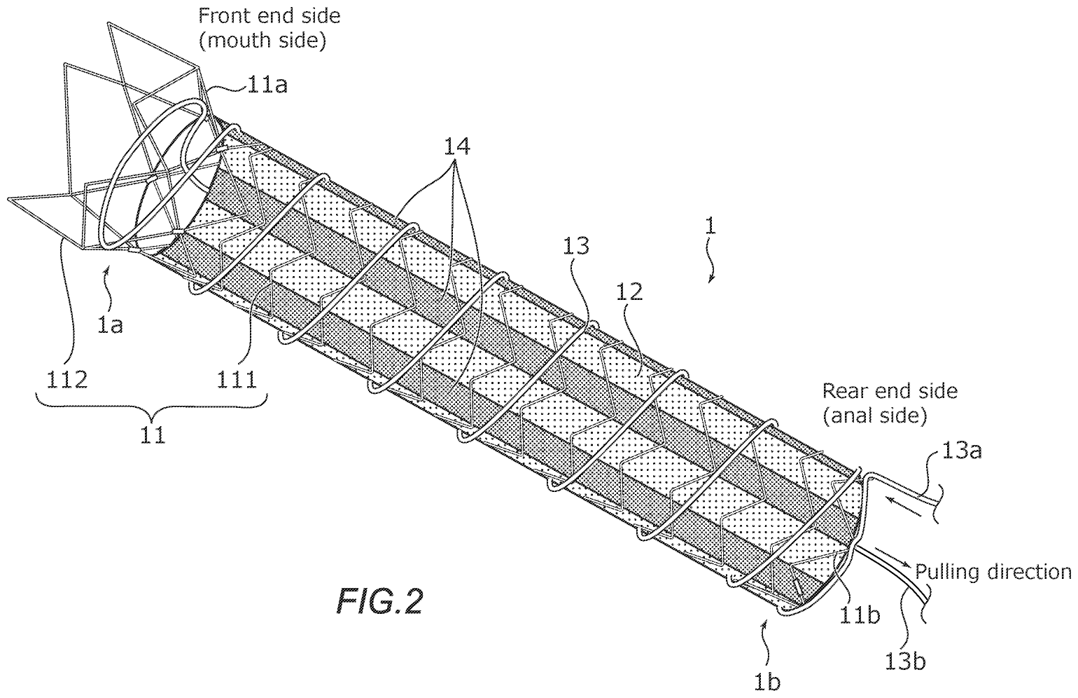

The large intestine stent 1 is attached to the holding portion 32 of the inner rod 3, and a restraint string 13 (described in detail later) is wound around the outer peripheral face of the large intestine stent 1. Both ends of the restraint string 13 are drawn out from e.g. a branch opening 22a provided on the hub 22. The configuration of the large intestine stent 1 will be described in detail with reference to FIG. 2.

FIG. 2 is a perspective view schematically illustrating the large intestine stent 1.

The large intestine stent 1 has a cylindrical shape that demarcates a tubular flow path through which digests flow. The large intestine stent 1 is a unilateral flare-type partially covered stent. The large intestine stent 1 is placed on the lesion site in the large intestine such that a flared end portion 1a is positioned on an upstream side (mouth side) of the digest flow direction and the other end portion 1b is positioned on a downstream side (anal side) of the digest flow direction. In the following description, the left side (flared end portion side) in FIG. 2 will be explained as the front end side, and the right side (straight end portion side) will be explained as the rear end side.

The large intestine stent 1 may have a removal auxiliary portion for hooking a removal wire on an end portion that is drawn into a recovery tube or the like during removal.

As illustrated in FIG. 2, the large intestine stent 1 includes a skeleton portion 11 and a membrane portion 12. In addition, the skeleton portion 11 is wound with a restraint string 13 that functionally acts as a conversion section for controlling the expanded/contracted state of the skeleton portion 11. Furthermore, the large intestine stent 1 has an extension restricting portion 14 arranged along the axial direction.

The skeleton portion 11 is a stiffening member for holding the membrane portion 12 in a predetermined expanded state. In addition, the skeleton portion 11 has a so-called self-expandability, in which an expanded shape is memorized. That means, the skeleton portion 11 (first skeleton portion 111 and second skeleton portions 112) is configured to be self-expandable in the radial direction substantially perpendicular to the axial direction, from a contracted state of contracting inward to an expanded state of expanding outward to demarcate a cylindrical flow path.

Examples of a material of the metal wire constituting the skeleton portion 11 include known metals or metal alloys typified by a stainless steel, a nickel-titanium alloy (Nitinol), a titanium alloy, and the like. Also, alloy materials having X-ray contrast property may be used. In this case, the position of the large intestine stent 1 can be confirmed from outside the body. The skeleton portion 11 may be made of a material other than metal materials (e.g. a ceramic, a resin, or the like).

In addition, the skeleton portion 11 (particularly, the first skeleton portion 111) is configured to be able to deform responding to an external force applied from the outer face side. As a result, even if the external force is applied to the large intestine stent 1 from the outer face side, the skeleton portion 11 deforms, so that the lesion site of the large intestine can be continuously widened while the stent stays at the placement site without causing kink.

The material, a wire type (e.g. a circular wire rod such as a wire, or an angular wire rod obtained by laser processing), a wire diameter (cross-sectional area), a number and a shape of folds in a circumferential direction (a number and a shape of crest portions), an interval of the wire rod in the axial direction (spiral pitch (amount of the skeleton per a unit length)), and the like of the wire rod constituting the skeleton portion 11 are appropriately selected the basis of flexibility required depending on the gastrointestinal tract to be placed. Flexibility refers to the ease of bending the large intestine stent 1, and is defined particularly by a flexural rigidity (synonymous with straightening force) in an axial direction. That is, the configuration in which the skeleton portion 11 can be deformed responding to the external force means that the skeleton portion 11 has a moderately low flexural rigidity in an axial direction and has a property of following the shape of the digestive tract or the sheath without causing kink in the digestive tract or the sheath.

In the present embodiment, the skeleton portion 11 has a cylindrical first skeleton portion 111 and the flared second skeleton portion 112 provided on one end side of the first skeleton portion 111. The large intestine stent 1 is placed in the large intestine such that the second skeleton portion 112 is positioned on the upstream side in the flow direction of the digests.

The first skeleton portion 111 is a main skeleton accounting for most of the skeleton portion 11. For example, the first skeleton portion 111 is formed in a cylindrical shape as a whole by spirally winding a metal wire rod while being bent such that crest portions and trough portions are alternately formed in the axial direction.

The second skeleton portion 112 is an end skeleton provided on the front end side of the first skeleton portion 111, and is formed e.g. by laser processing. The second skeleton portion 112 is connected to the first skeleton portion 111 e.g. by bringing a front end portion of the first skeleton portion 111 and a rear end portion of the second skeleton portion 112 into close contact with each other and caulking them.

The first skeleton portion 111 (excluding the rear end portion) is covered with the membrane portion 12. That means, the rear end portion of the first skeleton portion 111 and the second skeleton portion 112 are not covered with the membrane portion 12. Along the circumferential direction, the restraint string 13 is inserted into the rear end portion of the first skeleton portion 111 and the second skeleton portion 112. In addition, since the second skeletal portion 112 is not covered with the membrane portion 12, the second skeleton portion 112 makes inroads into a large intestinal wall when placing the large intestine stent 1 in the large intestine, so that displacement of the front end portion of the large intestine stent 1 can be suppressed.

In addition, the skeleton portion 11 is configured to be extendable and contractable in the axial direction while maintaining a radial-direction size. In the present embodiment, since the first skeleton portion 111 is formed by spirally winding the metal wire rod, the skeleton portion 11 is twisted in the spiral direction when extending and contracting in the axial direction, so that the radial-direction size of the skeleton portion 11 is maintained. As a result, even if the skeleton portion 11 extends and contracts in accordance with the deformation of the large intestine, the expanding force of the skeleton portion 11 is maintained, and therefore the lesion site of the large intestine can be widened while the stent stays at the placement site.

The membrane portion 12 is a film body that forms the flow path for the digests, and is arranged along the peripheral face of the skeleton portion 11. Since large intestinal wall cells can be prevented from exuding to the inside of the large intestine stent 1 by arranging the membrane portion 12 along the peripheral face of the skeleton portion 11, recurrence of the lesion (occlusion and stenosis) in the large intestine can be prevented.

The membrane portion 12 is formed by forming a film on a space composed of the wire rods constituting the skeleton portion 11 i.e. a peripheral face of the skeleton portion 11 e.g. using dipping. In addition, for example, the membrane portion 12 is made of a film material, and may be disposed on the outer peripheral face and the inner peripheral face of the skeleton portion 11 so as to sandwich the skeleton portion 11, or may be disposed on either the outer peripheral face or the inner peripheral face of the skeleton portion 11.

Examples of a material for forming the membrane portion 12 include a fluororesin such as a silicone resin and PTFE (polytetrafluoroethylene), a polyester resin such as polyethylene terephthalate, and the like.

The extension restricting portion 14 is composed of e.g. a tape-shaped elongated member, and fixed (e.g. by adhesion or the like) to the peripheral face (at least one of the inner peripheral face and the outer peripheral face) of the first skeleton portion 111 so as to extend over both end portions of the first skeleton portion 111 in an axial direction. In addition, for example, five extension restricting portions 14 are arranged at equal intervals in the circumferential direction.

The extension restricting portion 14 is formed of a biocompatible thread (e.g. polyester thread) or cloth (textile (fabric) or knit), and has a strength capable of restricting extension of the first skeleton portion 111 in the axial direction at least within a range that the expandability of the large intestine stent 1 in the radial direction is not impaired.

When the large intestine stent 1 is contracted in the radial direction and accommodated in the sheath 2, the extension of the large intestine stent 1 in the axial direction is suppressed by the extension restricting portion 14. Thereby, the large intestine stent 1 accommodated in the sheath 2 has a shorter length in an axial direction, and a contact area between the large intestine stent 1 and the sheath 2 is smaller, compared to a stent without the extension restricting portion 14. Thus, since a friction resistance in releasing the large intestine stent 1 from the sheath 2 becomes small, the large intestine stent 1 can be easily released from the sheath 2. In addition, when the large intestine stent 1 is released from the sheath 2 to expand, a reduction rate of the stent in an axial direction is decreased, so that the large intestine stent 1 can be placed at a desired placement site in the large intestine.

The restraint string 13 is made of a material having a predetermined strength and rigidity, and for example, a suture such as a nylon fiber and a fluorine fiber, a thin metal wire made of a nickel-titanium alloy or a stainless steel, or a string-shaped resin member can be applied. The restraint string 13 may be formed in a wide tape shape.

The restraint string 13 is wound around the outer peripheral face of the large intestine stent 1. Specifically, for example, on a rear end portion 11b of the skeleton portion 11 i.e. a part of the first skeleton portion 111 that is not covered with the membrane portion 12, the restraint string 13 is inserted into the first skeleton portion 111 along the circumferential direction so as to sew the first skeleton portion 111, and wound around the outer peripheral face of the first skeleton portion 111 e.g. in a spiral shape. In addition, the restraint string 13 extends to a front end portion 11a of the skeleton portion 11 i.e. the second skeleton portion 112, and is inserted into the second skeleton portion 112 along the circumferential direction so as to sew the second skeleton portion 112, and, for example, drawn out to the rear end side through the inside of the skeleton portion 11.

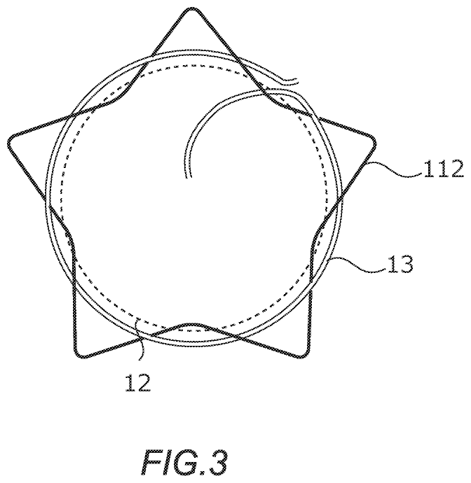

The phrase “inserted into the second skeleton portion 112 so as to sew the second skeleton portion 112” means that, for example, the restraint string 13 is inserted into the second skeleton portion 112 while crossing alternately the inside and the outside of the second skeleton portion 112 as illustrated in FIG. 3.

When the restraint string 13 is entwined with the front end portion 11a (second skeleton portion 112) and the rear end portion 11b (part of the first skeleton portion 111 that is not covered with the membrane portion 12) of the skeleton portion 11, the restraint string 13 is held on the front end portion 11a and the rear end portion 11b of the skeleton portion 11 so as not to drop off. Thereby, the restraint string 13 can be pulled out while maintaining the wound state, and the skeleton portion 11 can be released sequentially from one end side to expand. For example, when the restraint string 13 is pulled out by pulling the end portion drawn out to the rear end side through the inside of the skeleton portion 11, the skeleton portion 11 is released sequentially from the rear end portion 11b side to expand.

In this way, the restraint string 13 functionally acts as a conversion section that can convert the rear end portion 11b from the contracted state to the expanded state while maintaining the front end portion 11a of the skeleton portion 11 in the contracted state.

It is allowed to take a configuration that the restraint string 13 is not directly attached to the both end portions 11a and 11b of the skeleton portion 11, but a ring-shaped auxiliary member (not illustrated) is provided on the front end portion 11a and the rear end portion 11b of the skeleton portion 11, and the restraint string 13 is inserted into this auxiliary member.

When the large intestine stent 1 is attached to the holding portion 32 of the inner rod 3, the large intestine stent 1 is folded in the radial direction while extending in the axial direction so as to contract. At this time, one end portion 13b of the restraint string 13 is fixed and the other end portion 13a is pulled, so that looseness of the restraint string 13 accompanying the contraction of the large intestine stent 1 (skeleton portion 11) is absorbed. Thereby, the contracted large intestine stent 1 is tightly restrained by the restraint string 13. When the large intestine stent 1 is accommodated in the sheath 2, the both end portions 13a and 13b of the restraint string 13 are drawn outside from e.g. the branch opening 22a provided on the hub 22 (see FIG. 1A and FIG. 1B).

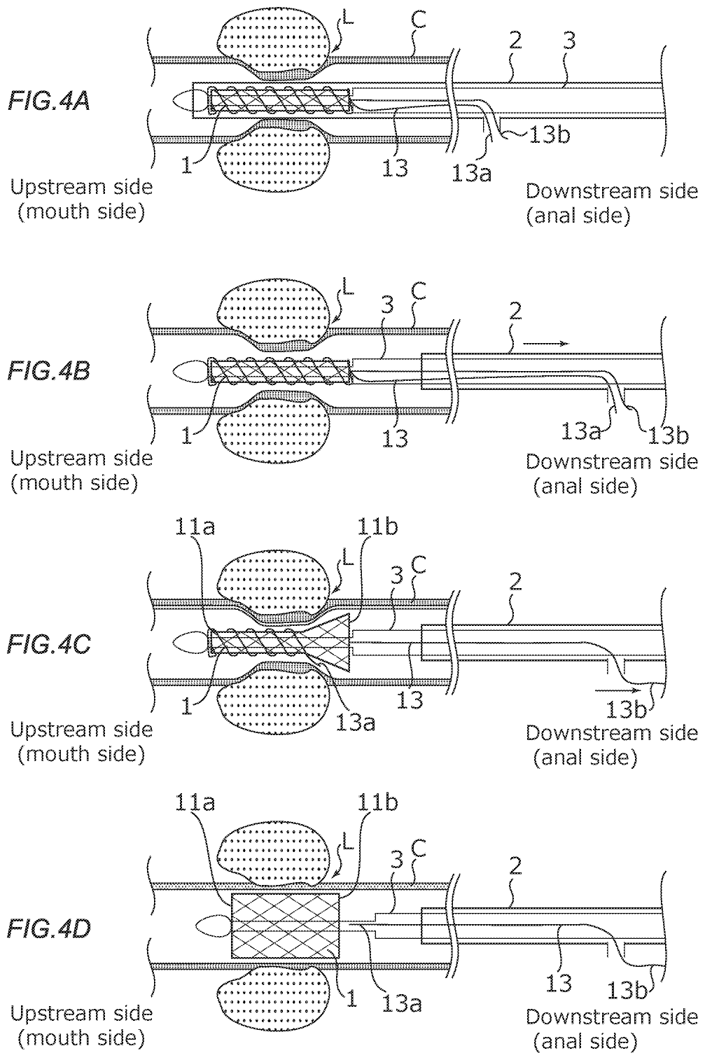

FIG. 4A to FIG. 4D are diagrams illustrating a state change during placement of the large intestine stent 1. FIG. 4A to FIG. 4D schematically illustrate the large intestine stent 1, and illustration of the flared part (second skeleton portion 112) not covered with the membrane portion 12 is omitted.

When the large intestine stent 1 is placed at a lesion site L (placement target site) of a large intestine C, the sheath 2 and the inner rod 3 are inserted from the anal side along a guide wire (not illustrated) previously introduced into the large intestine C, and the large intestine stent 1 is positioned at the lesion site L (see FIG. 4A).

Subsequently, the sheath 2 is moved to the anal side in the positioned state, and the large intestine stent 1 is released from the sheath 2 (see FIG. 4B). In the present embodiment, since the outer peripheral face of the large intestine stent 1 is restrained by the restraint string 13, the large intestine stent 1 is maintained while being contracted. The large intestine stent 1 may be released from the sheath 2 by moving the inner rod 3 so as to be pushed toward the mouth side while the position of the sheath 2 is fixed.

Subsequently, one end portion 13b (herein, end portion drawn out through the large intestine stent 1) of the restraint string 13 is pulled, and the restraint string 13 is gradually pulled out (see FIG. 4C). Since the other end portion 13a of the restraint string 13 passes through the rear end portion 11b of the skeleton portion 11 and moves to the distal end side, the large intestine stent 1 gradually shifts to the expanded state from the rear end side. At this time, the reduction rate of the large intestine stent 1 in an axial direction during expansion is decreased by the extension restricting portion 14, but the large intestine stent 1 is shortened in the axial direction, and therefore the placement position of the large intestine stent 1 is adjusted by handling the inner rod 3 forward and backward. Apart of the large intestine stent 1 (particularly, the front end portion 11a (second skeleton portion 112) of the skeleton portion 11 having a large expanding force (retention force)) is maintained in the contracted state by the restraint string 13, and is not completely expanded. Thus, even if the rear end portion 11b side of the skeleton portion 11 comes into contact with an inner wall of the large intestine C and presses the large intestine C in the radial direction, the placement position of the large intestine stent 1 can be easily adjusted.

Once the whole restraint string 13 is pulled out, the large intestine stent 1 is completely expanded, and the opening state of the large intestine C is ensured (see FIG. 4D). Subsequently, although not illustrated, the engagement between the large intestine stent 1 and the inner rod 3 is released, and the inner rod 3 is pulled out, so that the large intestine stent 1 is placed on the lesion site L. The placement position of the large intestine stent 1 is adjusted while gradually pulling out the restraint string 13, so that the large intestine stent 1 can be accurately positioned and placed at the lesion site L.

In this way, the large intestine stent 1 (gastrointestinal stent) according to the present embodiment has a cylindrical shape and includes the skeleton portion 11 expandable and contractable in the radial direction substantially perpendicular to the axial direction, and the restraint string 13 (conversion section) that can, while maintaining the front end portion 11a (one part) of the skeleton portion 11 in an axial direction in the contracted state, convert the rear end portion 11b (an other part) from the contracted state to the expanded state.

Thereby, a timing of expanding the front end portion 11a and the rear end portion 11b of the skeleton portion 11 can be controlled by the restraint string 13, the rear end portion 11b is expanded while maintaining the front end portion 11a in the contracted state, meanwhile the placement position of the large intestine stent 1 can be adjusted. That means, on the way of the placement during which only the front end portion 11a is expanded, the force of the large intestine stent 1 pressing the inner wall of the large intestine C becomes relatively small, so that the stent can be easily aligned, and the large intestine stent 1 can be accurately placed on the placement target site of the large intestine C.

In addition, in the large intestine stent 1, the restraint string 13 (conversion section) sequentially converts the skeleton portion 11 from the contracted state to the expanded state from the rear end portion 11b (an other part) corresponding to the downstream side (anal side) to the front end portion 11a (one part) corresponding to the upstream side (mouth side) in the flow direction of the digests.

As a result, for example, even in a case of placing the large intestine stent 1 on the anal neighborhood side of the large intestine C where accuracy of the placement position is required, the rear end portion 11b on the anal side can be expanded while maintaining the front end portion 11a on the mouth side in the contracted state, meanwhile the placement position of the large intestine stent 1 can be adjusted, so that the large intestine stent 1 can be accurately placed at the placement target site of the large intestine C.

In addition, the conversion section is the restraint string 13 (string-shaped member) wound around the skeleton portion 11. Thereby, the placement position can be adjusted while pulling out the restraint string 13 to gradually expand the skeleton portion 11, and the skeleton portion 11 can be converted from the contracted state to the expanded state by a very simple procedure i.e. the pulling out of the restraint string 13. In addition, since practitioners themselves can control the conversion amount, the placement operation can be proceeded at their own pace while confirming the contracting of the large intestine stent 1.

In addition, the restraint string 13 is held on the front end portion 11a and the rear end portion 11b (both end portions in the axial direction) of the skeleton portion 11 so as not to drop off. Thereby, only by pulling out the restraint string 13, for example, the skeleton portion 11 can be converted from the contracted state to the expanded state from the rear end portion 11b to the front end portion 11a of the skeleton portion 11.

As described above, the invention made by the present inventors has been specifically explained on the basis of the embodiment, but the present invention is not limited to the above embodiment, and can be modified without departing from the gist of the invention.

Although the case where the restraint string 13 is applied as the conversion section according to the present invention has been explained in the embodiment, any conversion section is allowed as long as the large intestine stent 1 can be maintained in the contracted state and gradually converted into the expanded state.

For example, a biocompatible and soluble wire rod or tape material may be wound around the skeleton portion 11 so as to functionally act as the conversion section. In this case, the large intestine stent 1 expands from a part where the conversion section is dissolved. For example, when the conversion section has different wire rod densities in the axial direction of the large intestine stent 1, from which part the stent 1 is shifted to the expanded state can be controlled.

In addition, although the case where the large intestine stent 1 expands from the downstream side (anal side) to the upstream side (mouth side) in the flow direction of the digests has been explained in the embodiment, the large intestine stent 1 may expand from the upstream side to the downstream side in the flow direction, or expand from a mid portion of the large intestine stent 1 toward the both end portions 1a and 1b. In both cases, it is preferable that a part where the expanding force of the large intestine stent 1 i.e. the retention force in the large intestine is highest is set so as to finally expand.

In addition, although the case where the first skeleton portion 111 has a straight cylindrical shape has been described in the embodiment, the case is merely an example and the present invention is not limited to this case. For example, the first skeleton portion 111 may have a curved shape depending on the placement site, or a curved shape along the shape of the digestive tract after the placement.

In addition, although the first skeleton portion 111 is formed by spirally winding the metal wire rod in the embodiment, the first skeleton portion 111 may be formed by weaving the metal wire rod or by laser processing. However, since the laser-cut type stent has a high flexural rigidity and a large straightening force, care must be taken not to impair the deformation responding to the external force.

In addition, in the embodiment, as the large intestine stent 1, the unilateral flare-type partially covered stent has been explained as an example, but this stent is merely an example, and the present invention is not limited to this stent and can be arbitrarily modified as appropriate.

The present invention can also be applied to e.g. a unilateral flare-type bare stent or fully covered stent, a bilateral flare-type partially covered stent, bare stent, or fully covered stent, and a straight-type fully covered stent or bare stent. Although not illustrated, in the case of the covered stent with the skeleton portion 11 covered with the membrane portion 12, the restraint string 13 may be attached to the membrane portion 12 so as to sew the membrane portion 12.

Furthermore, the present invention can be applied not only to the large intestine stent explained in the embodiment but also to a gastrointestinal stent that is placed in a gastrointestinal tract such as esophagus and bile duct. In this case, a fluid flowing through the digestive tract includes e.g. a food immediately after intake, which has not been digested at all, a decomposed food that has passed through the digestive tract, a matter that has not been digested even after going through the digestive tract (e.g. stool, or the like), and the like, regardless of a state of the matter.

The embodiment disclosed in this specification is an example in all regards and should be regarded as unrestrictive. The scope of the present invention is stipulated not by the aforementioned explanation but by claims, and intended to include meanings equivalent to claims, and all modifications within the scope of claims.

Disclosure contents of specifications, figures, and abstracts included in Japanese Patent Application No. 2018-233628 filed on Dec. 13, 2018 are all incorporated in this application.

### DESCRIPTION OF REFERENCE NUMERALS

1 Large intestine stent (gastrointestinal stent)
11 Skeleton portion
11a Front end portion (one part)
11b Rear end portion (an other part)
13 Restraint string (conversion section, string-shaped member)
C Large intestine

1. A gastrointestinal stent, which is placed in a gastrointestinal tract, the gastrointestinal stent comprising:
a skeleton portion having a cylindrical shape and capable of expanding and contracting in a radial direction substantially perpendicular to an axial direction; and
a conversion section that is capable of, while maintaining one part of the skeleton portion in the axial direction in a contracted state, converting an other part from the contracted state to an expanded state.

2. The gastrointestinal stent according to claim 1, wherein the conversion section sequentially converts the skeleton portion from the contracted state to the expanded state from the other part corresponding to a downstream side to the one part corresponding to an upstream side in a flow direction of digests.

3. The gastrointestinal stent according to claim 1, wherein the conversion section is a string-shaped member wound around the skeleton portion.

4. The gastrointestinal stent according to claim 3, wherein the string-shaped member is held on both end portions of the skeleton portion in the axial direction so as not to drop off.
